(19) United States
(12) Patent Application Publication
(10) Pub. No.: US 2013/0203602 A1
(43) Pub. Date: Aug. 8, 2013

(54) SUPPORTED BIOLOGICALLY ACTIVE COMPOUNDS

(71) Applicants: Anders Riisager, Tastrup (DK); Rasmus Fehrmann, Copenhagen (DK); Hector Rodriguez, Teo (ES); Katharina Bica, Vienna (AT); Robin D. Rogers, Tuscaloosa, AL (US); Daniel T. Daly, Tuscaloosa, AL (US); Gabriela Gurau, Tuscaloosa, AL (US)

(72) Inventors: Anders Riisager, Tastrup (DK); Rasmus Fehrmann, Copenhagen (DK); Hector Rodriguez, Teo (ES); Katharina Bica, Vienna (AT); Robin D. Rogers, Tuscaloosa, AL (US); Daniel T. Daly, Tuscaloosa, AL (US); Gabriela Gurau, Tuscaloosa, AL (US)

(73) Assignees: Danmarks Tekniske Universitet, Kongens Lyngby (DK); The Board of Trustees Of The Univ. Of Alabama For And On Behalf Of The Univ. Of Alabama, Tuscaloosa, AL (US); The Queens University of Belfast, Belfast (GB)

(21) Appl. No.: 13/583,890

(22) PCT Filed: Mar. 11, 2011
(86) PCT No.: PCT/EP11/53696

§ 371 Date: Dec. 20, 2012

Related U.S. Application Data
(60) Provisional application No. 61/313,084, filed on Mar. 11, 2010.

(30) Foreign Application Priority Data
Mar. 11, 2010 (EP) 10156242.9

(51) Int. Cl. A01N 25/08; A61K 9/10

(52) U.S. Cl. CPC A01N 25/08; A61K 9/10
USPC 504/358; 424/400; 514/263.38; 514/254.07; 514/570

(57) ABSTRACT

The present invention relates to biologically active compounds, particularly liquid compounds, which are immobilized on a solid carrier material, particularly on mesoporous silica. The compounds are non-covalently supported on the solid carrier material thereby forming stable, easily handled solids which have the further advantage that the adsorbed biologically active compounds have improved thermal stability compared with the non-adsorbed compounds, and that they are released rapidly and completely from the carrier material when placed in an aqueous environment.

### BACKGROUND

In recent times, there has been a notable interest in the design of active pharmaceutical ingredients (API) and biologically active compounds in liquid form, since the liquid state can have a profound impact on important properties for successful drug development (Hough et al., New J. Chem. 2007, 31, 1429-1436; Hough and Rogers, Bull. Chem. Soc. Jpn. 2007, 80, 2262-2269). Liquid strategies can take advantage of the dual nature (discrete ions) of liquid salts (ionic liquids, molten salts) to realize enhancements which may include controlled solubility (e.g., both hydrophilic and hydrophobic ionic liquids are possible), bioavailability or bioactivity, stability, elimination of polymorphism, new delivery options or even customized pharmaceutical cocktails (Rogers et al., WO 2007044693). However, the liquid state properties also have significant impact on ease of preparation and handling compared to solid drugs, and need special devices for dosing and administration.

In existing methods and systems, the biologically active liquids are supported on porous solid bodies such as mesoporous silica. Typically, the liquids are covalently attached to the solid bodies. The functionalized organic groups present in the liquids are covalently bonded to one or more pores of the solid carrier material. For example, in the case of mesoporous silica carrier material, the mesoporous silica body contains a room temperature ionic liquid (RTIL) such as an antimicrobial agent (Lin et al., US 20060018966). According to Pavlin et al, WO2008079892, matrix-immobilized active liquids release the active ingredient into the ambient environment.

The groups of Fehrmann and Wasserscheid introduced the concept of Supported Ionic Liquid Phase (SILP) catalysts for the immobilization of a transition metal catalyst dissolved in ionic liquids on solid carrier material (Riisager et al., Eur. J. Inorg. Chem. 2006, 695-706). In these SILP systems, a thin film of ionic liquid containing the homogeneous catalyst is immobilised on the surface of a high-area, porous carrier material. Consequently, SILP catalyst systems offer significant advantages compared to biphasic catalysis in organic liquid/ionic liquid mixtures. Examples of transition metal catalyzed reactions include hydroformylation, carbonylation, hydrogenation, Heck reactions, hydroaminations and epoxidation.

Biologically active molecular species such as enzymes have previously been immobilized onto hydrophobic porous polymeric materials by hydrophobic-hydrophobic interactions [E. Ruckenstein and X. Wang, Biotech. and Bioeng., Vol 42 pg 821 (1993); Thies et al., US 20090215913]. This physisorption is non-covalent and while the biologically active molecular species (enzyme) retains some of its activity, the nature of the physisorption is such that the biologically active molecular species can be removed (leached) from the polymeric carrier material and therefore the activity of the system drops with subsequent reuse. This can also be seen for commercial systems where enzymes have been immobilized onto polymer beads via non-covalent physisorption processes, such as Novozyme 435. However, these enzymes immobilized onto the hydrophobic porous materials have been used as biocatalyst.

The same principle of adsorption onto high surface area carriers is a well known technique to enhance drug dissolution, and has already been described for inorganic silica, carbon materials and layered double hydroxides as well as polymeric matrices as solid carrier material (Cavallaro et al., Drug Deliv. 2004, 11, 41-46). Mesoporous silica-based systems have attracted particular attention for the controlled delivery of drugs as they are non-toxic, biocompatible and bioerodible (Wang et al., Microporous Mesoporous Mater. 2009, 117, 1-9). Methods for preparing a series of mesoporous silicates, such as RTIL-templated mesoporous silicate particles, with various particle morphologies are provided. The room-temperature ionic liquid is an antimicrobial agent within the pores of silicate particles. The particles can be used as controlled-release nanodevices to deliver antimicrobial agents (Lin et al., US 20060018966).

None of the reports in the field of supported biologically active liquids describe the non-covalent interaction of biologically active liquids (ionic or non-ionic) with the solid carrier material, especially with pre-formed solid carrier materials. This interaction is highly interesting as regards the future development of controlled release formulations based on adsorbing biologically active liquids and other biologically active compounds in general on solid carrier material, as both the nature of the carrier material (such as for example surface topology, porosity and chemical composition) and the nature of the biologically active compound itself (such as, for example, presence or absence of ionic components and the compounds hydrophilicity or hydrophobicity) are expected to influence the release rate of the adsorbed compound from the carrier material, thereby making it possible to tailor-make biologically active compositions matching very different requirements.

Finally, the complete and rapid release of such biologically active liquids supported on solid carrier material when placed in an aqueous environment as demonstrated herein has so far not been disclosed either.

### SUMMARY OF THE INVENTION

Accordingly, the present invention provides a biologically active composition comprising a biologically active compound, in particular a biologically active liquid compound, which is non-covalently and releaseably adsorbed or supported on, or attached to a solid carrier material, wherein by placement in an aqueous environment said biologically active compound is released from said carrier material.

The solid carrier material is preferably mesoporous silica. An enhanced thermal stability of the adsorbed biologically active compound was observed when compared with the non-adsorbed compound. The biologically active compound is released from the solid carrier material when placed into an aqueous environment, such as simulated gastric fluid or simulated intestinal fluid.

According to an embodiment of the invention, a composition comprising the biologically active composition is also provided which may be employed as a pharmaceutical, pesticidal, veterinary or agrochemical composition, or simply as a practical, easier handled solid form of a liquid compound.

### BRIEF DESCRIPTION OF THE FIGURES

FIG. 1 depicts the thermal stability of silica-supported Acyclovir using thermogravimetrical analysis (TGA).

FIG. 2 depicts the thermal stability of silica-supported Choline Acyclovir [1] using thermogravimetrical analysis (TGA).

FIG. 3 depicts the thermal stability of silica-supported Tributylmethyl-ammonium Acyclovir [2] using thermogravimetrical analysis (TGA).

FIG. 4 depicts the thermal stability of silica-supported Trimethylhexadecyl-ammonium acyclovir [3] using thermogravimetrical analysis (TGA).

FIG. 5 depicts the thermal stability of silica-supported Dioctylsulfosuccinic Acid [4] using thermogravimetrical analysis (TGA).

FIG. 6 depicts the thermal stability of silica-supported Itraconazolium Dioctylsulfosuccinate [5] using thermogravimetrical analysis (TGA).

FIG. 7 depicts the thermal stability of silica-supported Tetraethylammonium Glyphosate [6] using thermogravimetrical analysis (TGA).

FIG. 8 depicts the thermal stability of silica-supported Ibuprofene using thermogravimetrical analysis (TGA).

FIG. 9 depicts the thermal stability of silica-supported Tetrabutylphosphonium Ibuprofenate [7] using thermogravimetrical analysis (TGA).

FIG. 10 depicts the leaching kinetics of silica-supported tetrabutylphosphonium ibuprofenate [7] with different loading in phosphate buffered saline (PBS) at pH 7.4.

### DEFINITIONS

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OA¹ where A¹ is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and hetero-cycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

### DETAILED DESCRIPTION OF THE INVENTION

It has now been found that liquid biologically active compounds, such as for example, some ibuprofenate salts can be adsorbed or supported non-covalently on a solid carrier material such as mesoporous silica and certain other inorganic, carbonaceous or polymeric carrier materials, and thereby be transformed into a solid compound with improved thermal stability and ease of handling and dosing. Such supported biologically active compounds (including pharmaceutically active compounds) can be easily released from the solid carrier material when placed in an aqueous environment, such as for example simulated gastric fluid or simulated intestinal fluid.

Further, the solid carrier material is insoluble in water, thereby providing the advantages of a solid drug form.

“Biologically active liquids”, or alternatively “liquid biologically active compounds” as referred to in the present invention include liquid compounds having controlling and/or curative effects in a biological system. Examples of the biologically active liquid as used to disclose the present invention include any kind of synthetic drug or molecule with biological, pharmaceutical or pharmacological activity including but not limited to therapeutic drugs, pesticides, insecticides, fungicides and the like. The biologically active liquid may also include dual functional ionic liquids, the constituents of which, in combination, can achieve improved activity or synergistic effects.

The biologically active compound (hereinafter interchangeably used as biologically active liquid) is preferably in liquid state at or below the human body temperature, preferably having a melting or glass transition point below 37 degree Celsius or even more preferably below 25 degree Celsius. In certain cases or for certain applications it may however be advantageous to employ biologically active liquids having a melting or glass transition point above 37 degree Celsius.

The term “liquid compound” or “biologically active liquid” as used herein includes a single compound or a mixture of two or more compounds, such as a eutectic mixture. Herein, the term “eutectic” means a mixture of two or more compounds which has a lower melting temperature than any of its individual compounds. The eutectic mixture is typically composed of non-ionic compounds, ionic compounds or mixtures thereof.

The liquid can be an ionic or non-ionic liquid. The liquid may contain one, two or more different components of which one or more may be ionic compounds such as salts. For example, the biologically active cations can be selected from differently substituted sulfonium, phosphonium or ammonium ions, or mixtures thereof, such as:

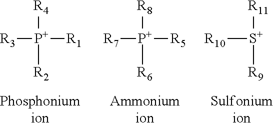

wherein R1, R2, R3, R4, R5, R6, R7, R8, R9, R10 and R11 can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above. The positively charged P, N and S atoms may also individually be part of heterocyclic or heteroaromatic structures by letting, e.g., R1 and R2 be fused such that a cyclic phosphonium ion is formed. Likewise, by letting eg. R5 and R6 be fused, a cyclic ammonium ion is formed, typical examples of which would be pyridinium and imidazolium. Finally, by letting eg. R9 and R10 be fused, a cyclic sulfonium ion is formed.

For other examples of biologically active compounds comprised by the present invention and their components, see table 1-3.

The liquid compound is supported onto the solid carrier material. The solid carrier material is substantially or completely insoluble in water, preferably porous, and provides a medium to hold the liquid. The liquid compound is non-covalently adsorbed on its surface including the porous structure of the solid carrier material. The solid carrier material should preferably be a pharmaceutically acceptable and substantially non-toxic material, which can be any one of an inorganic, carbonaceous, and polymeric carrier materials. Preferably, the solid carrier material is mesoporous silica with large surface area and pore volume, a highly ordered pore structure and adjustable pore size. In alternate embodiments of the present invention, porous synthetic foam, porous ceramic, activated carbon, diatomaceous earth, zeolites, kieselguhr, charcoal, porous alumina, porous titania, porous zirconia, porous silica or clay is employed. Other carbon materials or layered double hydroxides can also be used as a solid carrier material for the liquid.

The solid carrier material provides improved ease of handling, thermal stability and controlled release of the biologically active liquid compounds. The supported liquid compound furthermore has advantages over traditional solid drug forms, such as the elimination of polymorphism and the potential to control and improve physical properties such as melting point, solubility and rate of dissolution of the active compound.

In one embodiment of the invention, the polymeric carrier material is selected from any one of poly (N-isopropylacrylamide) and alkyl vinyl ether-maleic copolymer or poly (lactic acid).

### Adsorption on the Solid Carrier Material

The adsorption of a biologically active liquid compound on a particular solid carrier material is accomplished by dissolving the biologically active liquid compound in a suitable solvent and stirring the resulting solution with the solid carrier material for a sufficient period of time to allow equilibrium inside the pores to be established by pore diffusion (typically a couple of hours), evaporating the solvent slowly and removing the last traces of solvents in vacuo. The resulting solid material is easier to handle than the biologically active liquid compound itself, which can often be quite viscous, and can be prepared (“loaded”) with a high degree of precision.

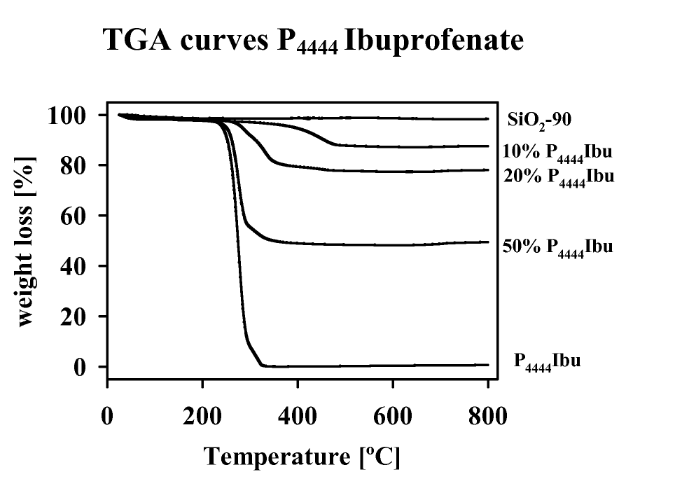

The present invention thus provides a methodology to adsorb a biologically active ionic liquid on a solid carrier material such as eg. mesoporous silica to improve handling of the viscous liquid and optionally to facilitate dosing while still keeping its originally liquid state. Due to the mesoporous structure of the used silica carrier material, the adsorbed ionic liquid can be obtained as a solid material even in high loading of 50% (wt/wt) (cf. FIG. 9).

### Improved Stability of the Supported Biologically Active Liquid Compound

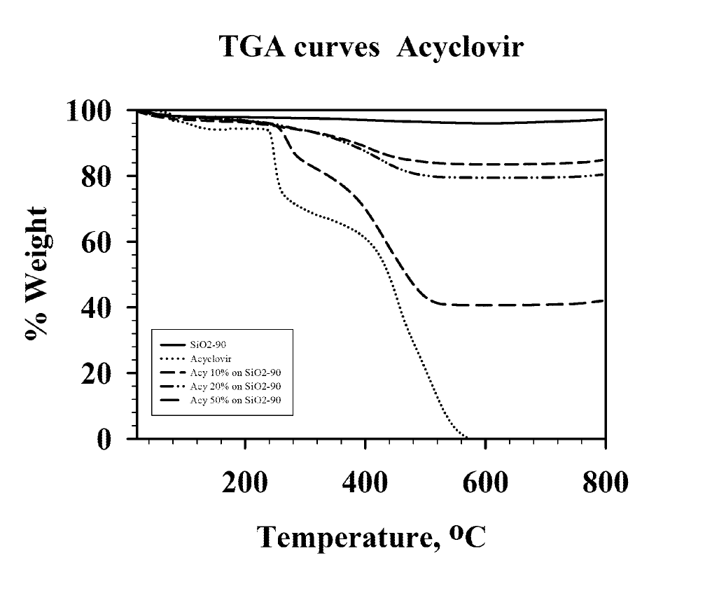

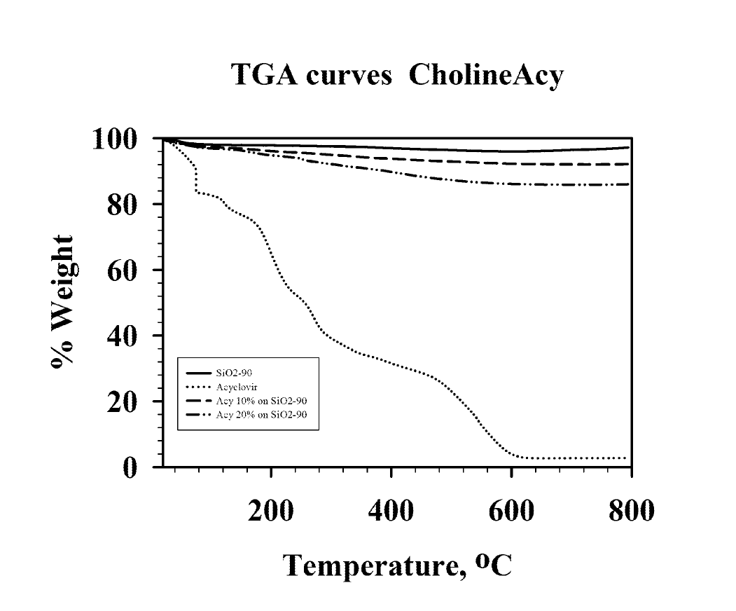

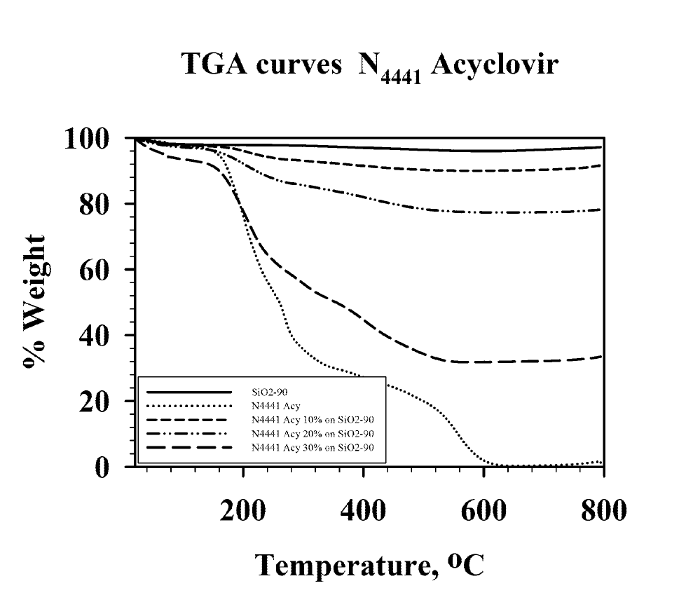

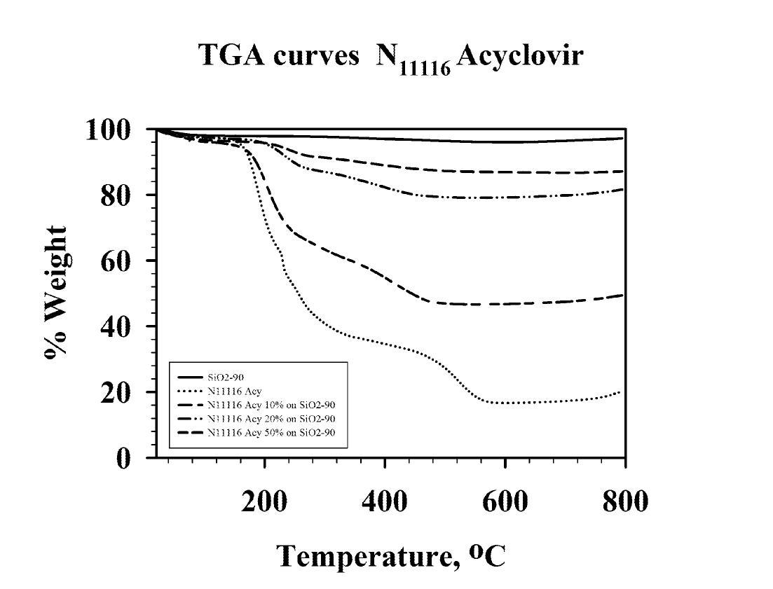

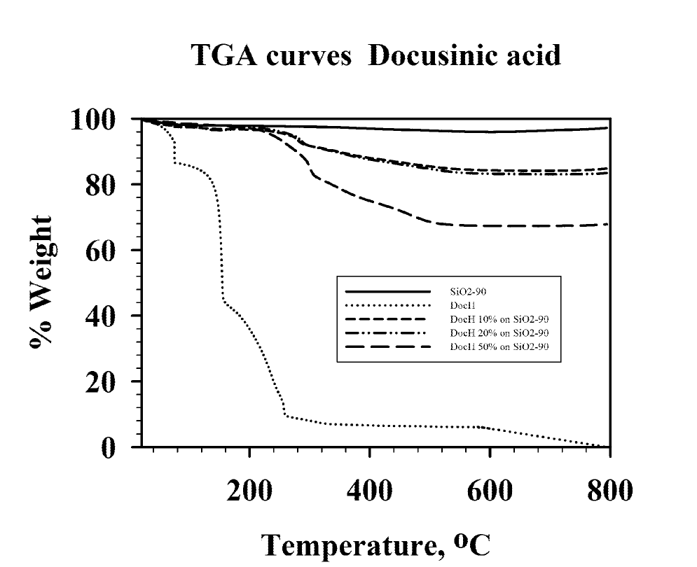

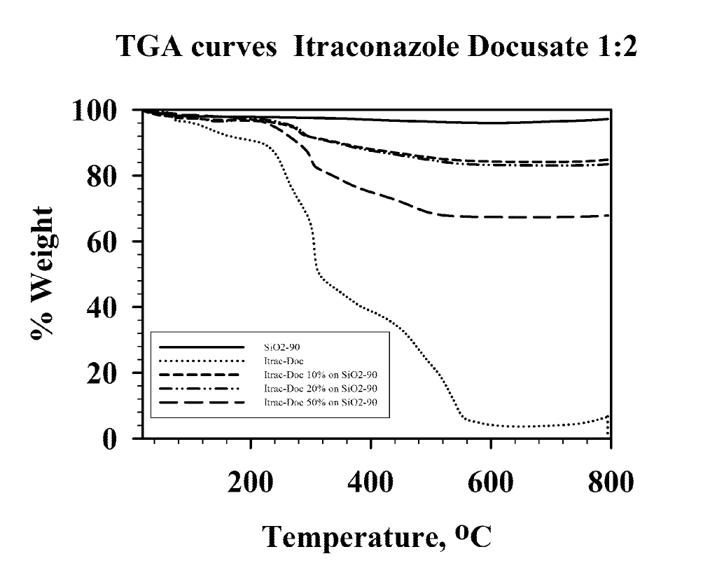

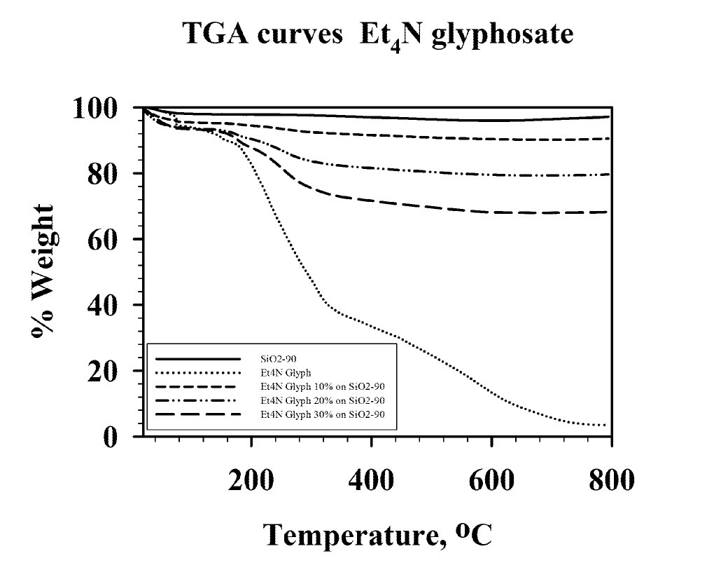

Apart from the easier handling discussed above, the adsorption of the biologically active liquid compound on certain solid carrier materials have surprisingly shown other advantages as well. It has thus been found that silica-supported biologically active liquids have considerably improved thermal stability compared to the pure (neat) liquids, and this effect has also been observed for certain other solid carrier material such as porous alumina. For example, the thermal degradation onset temperature of 10% (wt/wt) tetrabutylphosphonium ibuprofenate adsorbed on silica (T_{5%onset} 386° C.) is 150° C. higher than the one of the pure ionic liquid tetrabutylphoshonium ibuprofenate (T_{5%onset} 236° C.) (Table 4, FIG. 9). A number of the other investigated supported biologically active liquids also display enhanced thermal stability, even on higher loadings cf. Table 4 and FIGS. 1-10.

Without wishing to be bound by a specific scientific explanation for this behaviour, it is assumed that the increased thermal stability is due to hydrogen bonds established between hydrogen donor moieties on the adsorbed compound and hydrogen bond receptor sites on the surface of the porous silica, and conversely also due to hydrogen bonds established between hydrogen acceptor moieties on the adsorbed compound and acidic sites on the surface of the porous silica.

The silica-supported biologically active compounds may well also be more resistant to oxidation than the unsupported liquids.

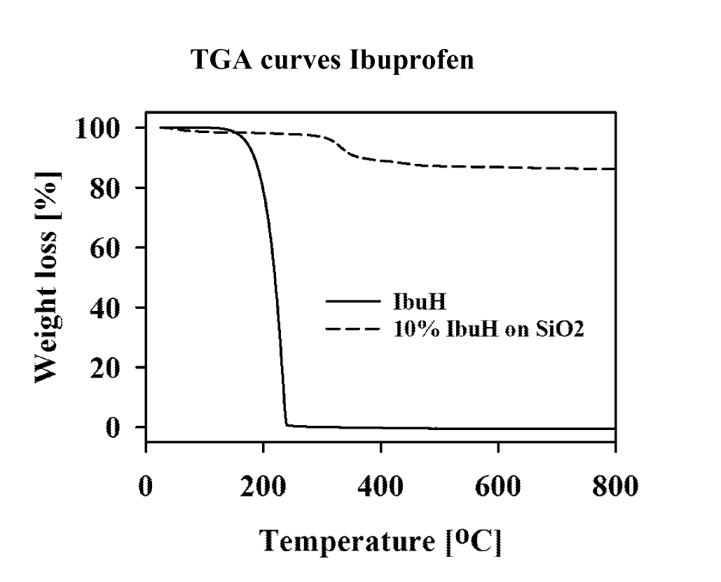

The above discussed phenomena of increased stability also apply to a range of adsorbed biologically active compounds which are higher melting solids. Ibuprofen, for example, has a m.p. of 74-78° C. When heated “neat” it starts to decompose around 150° C., whereas Ibuprofen adsorbed on silica first starts to decompose around 300° C. (FIG. 8 and Table 4).

Acyclovir is another example. Acyclovir has a m.p. of 256° C. When heated “neat” it starts to decompose around 249° C., whereas Acyclovir adsorbed at a loading of 50% on silica first starts to decompose around 270° C. (FIG. 1 and Table 4).

The present invention thus provides a methodology to improve the stability of biologically active compounds by adsorption on a solid carrier material, in particular the thermal stability of biologically active liquid compounds.

The present invention also provides the use of mesoporous silica to enhance the thermal stability of biologically active compounds adsorbed on said mesoporous silica.

Release of the Supported Biologically Active Compounds from the Carrier Material

The adsorption of biologically active compounds on solid carrier materials according to the present invention takes place in a reversible or releaseable manner, such that by placing the “loaded” carrier material in an aqueous environment such as, for example, simulated gastric fluid or simulated intestinal fluid, the supported biologically active liquids (including pharmaceutically active compounds) are released rapidly and completely from the carrier material. As another example of aqueous environments can be mentioned wet or moist soil, which is a relevant environment for agrochemical and/or pesticidal uses of the products of the invention. As yet another example can be mentioned aqueous beverages such as water, milk, tea or juice, which are relevant environments when using the products of the invention as solid forms of liquid drugs which must be dissolved prior to use. As yet another example can be mentioned water or aqueous solutions of chemicals, to which is added a product of the invention, thereby producing a final aqueous composition or solution which can be sprinkled on plants or soil.

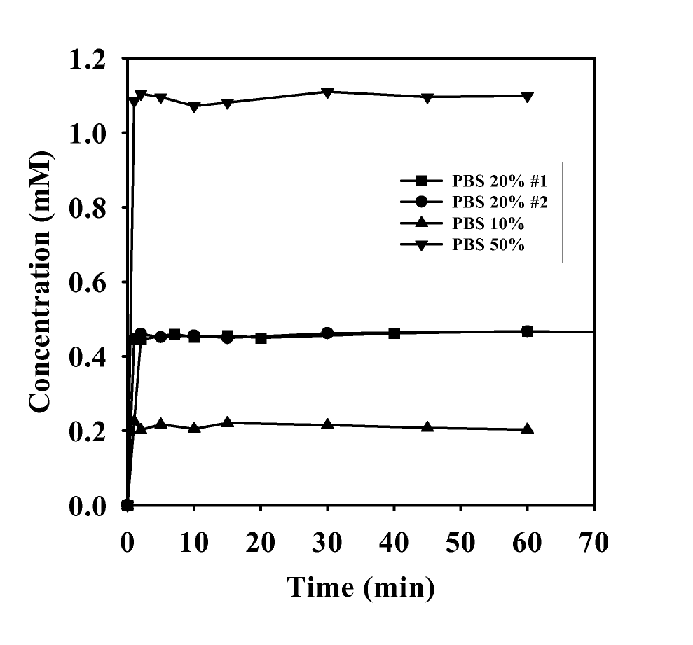

The rate of release is very fast. For example, the release from silica-supported tetrabutylphosphonium ibuprofenate loaded with different concentrations was examined in phosphate buffered saline at pH 7.4 (FIG. 10). FIG. 10 displays the release kinetics of silica-supported tetrabutylphosphonium ibuprofenate at a 50% initial load giving a 1.1 mM concentration in phosphate buffered saline (PBS), two runs at a 20% initial load giving a 0.45 mM concentration in PBS and at a 10% initial load giving a 0.2 mM concentration in PBS. In all cases the release was complete within a few minutes.

The release may well be dependent on both external factors such as pH of the aqueous environment, and internal factors such as chemical composition and surface topology of the solid carrier material including pore size, porosity, pore distribution and micro pH or charge of the pore surface. Finally, the physicochemical characteristics of the biologically active liquid also play a role such as its ionic/non-ionic nature and its hydrophilicity/hydrophobicity.

One of the key benefits of supported ionic liquid phase (SILP) delivery systems is the ability to control and fine-tune the release of the adsorbed ionic liquid by adjusting the design of the ionic liquid form (i.e. the choice of anion and cation) of the active compound and/or by adjusting the solid carrier material. The flexibility of the supported ionic liquid phase (SILP) drug delivery technology thereby offers wide possibilities to design future tailor-made drug formulations.

The present invention thus provides the use of a biologically active liquid composition for drug delivery and in-vitro release from a solid carrier material with rapid and complete release in an aqueous environment, such as, for example, simulated gastric fluid or simulated intestinal fluid.

In a first aspect the present invention therefore provides a composition comprising a biologically active compound which is non-covalently and releaseably adsorbed on a solid carrier material, wherein, by placement in an aqueous environment, said biologically active compound is released from said carrier material.

In a preferred embodiment the biologically active compound is in a liquid state. In an even more preferred embodiment the biologically active compound is in a liquid state at or below the human body temperature, preferably having a melting or glass transition point below 37 degree Celsius or even more preferably below 25 degree Celsius. In certain cases or for certain applications it may however be advantageous to employ biologically active compounds having a melting or glass transition point above 37 degree Celsius.

In a specific embodiment the aqueous environment wherein the composition of the invention is placed, and wherein the biologically active liquid is released, is gastric fluid.

In another specific embodiment the aqueous environment wherein the composition of the invention is placed, and wherein the biologically active liquid is released, is intestinal fluid.

In another specific embodiment the aqueous environment wherein the composition of the invention is placed, and wherein the biologically active liquid is released, is saliva.

In another specific embodiment the aqueous environment wherein the composition of the invention is placed, and wherein the biologically active liquid is released, is moist or wet soil.

In another specific embodiment the aqueous environment wherein the composition of the invention is placed, and wherein the biologically active liquid is released, is an aqueous beverage or infusion such as water, milk, fruit juice, tea or the like.

In a specific embodiment, the present invention provides compositions according to the first aspect of the invention wherein the biologically active compound comprises one or more compounds and mixtures thereof.

In a further embodiment, the present invention provides compositions according to the first aspect of the invention wherein the biologically active compound comprises mixtures of ionic and non-ionic compounds.

In another embodiment, the present invention provides compositions according to the first aspect of the invention wherein the biologically active compound is a eutectic mixture comprising one or more biologically active compounds.

In another embodiment, the present invention provides compositions according to the first aspect of the invention, wherein the biologically active compound is a mixture of oligomers, liquid ion pairs, hydrates, solvates or partially ionized species.

In another embodiment, the present invention provides compositions according to the first aspect of the invention wherein the biologically active compound is an ionic liquid comprising oligomeric cations or anions composed of one or more biologically active compounds.

In another embodiment, the present invention provides compositions according to the first aspect of the invention wherein the biologically active compound is an ionic liquid comprising liquid ion pairs that are ion paired to the extent of i) 75% to 100%; ii) 50% to 100%; iii) 5% to 100% in neat form or when placed in solutions.

In another embodiment, the present invention provides compositions according to the first aspect of the invention wherein the biologically active compound is an ionic liquid comprising partially ionized biologically active compounds with a degree of ionization of i) 75% to 100%; ii) 50% to 100%; iii) 5% to 100% and iv) 1% to 100% in neat form or when placed in solutions.

In a specific embodiment, the present invention provides compositions according to the first aspect of the invention wherein the biologically active compound is an ionic liquid comprising solvated biologically active compounds and various amounts of solvent involved in direct solvation, thereby forming ionic liquid solvates. If the chosen solvent is water, said solvates are ionic liquid hydrates.

In another embodiment, the present invention provides compositions according to the first aspect of the invention wherein the biologically active compound is non-ionic.

In another embodiment, the present invention provides compositions according to the first aspect of the invention wherein the biologically active compound is liquid at or below about 25° C.

In another embodiment, the present invention provides compositions according to the first aspect of the invention wherein the biologically active compound is liquid at or below about 37° C.

In a second aspect, the present invention provides compositions according to the first aspect of the invention wherein the biologically active compound comprises one or more biologically active ions, such as one or more biologically active cations and/or one or more biologically active anions.

In another embodiment, the present invention provides compositions according to the second aspect of the invention, wherein by placement in an aqueous environment both the anionic and cationic parts of said ionic compound are released from said carrier material.

In another embodiment, the present invention provides compositions according to the second aspect of the invention wherein the biologically active compound comprises one or more antibacterial, antiviral, antifungal, anti-inflammatory or pain relieving compounds.

In another embodiment, the present invention provides compositions according to the second aspect of the invention wherein the one or more biologically active cations is a pharmaceutically active compound, and the one or more biologically active anions is a taste modifier, or wherein the one or more biologically active cations is a taste modifier and the one or more biologically active anions is a pharmaceutically active compound.

In another embodiment, the present invention provides compositions according to the second aspect of the invention wherein the one or more biologically active cations is an antibacterial and the one or more biologically active anions is a taste modifier, or wherein the one or more biologically active cations is a taste modifier and the one or more biologically active anions is an antibacterial.

In another embodiment, the present invention provides compositions according to the second aspect of the invention wherein the one or more biologically active cations is an antibacterial and the one or more biologically active anions is a pain reliever or anti-inflammatory, or wherein the one or more biologically active cations is a pain reliever or anti-inflammatory and the one or more biologically active anions is an antibacterial.

In another embodiment, the present invention provides compositions according to the second aspect of the invention wherein the one or more biologically active cations is an anesthetic and the one or more biologically active anions is an antibacterial, or wherein the one or more biologically active cations is an antibacterial and the one or more biologically active anions is an anesthetic.

In another embodiment, the present invention provides compositions according to the second aspect of the invention wherein the one or more biologically active cations is a pain reliever and the one or more biologically active anions is an anti-inflammatory, or wherein the one or more biologically active cations is an anti-inflammatory and the one or more biologically active anions is an pain reliever.

In another embodiment, the present invention provides compositions according to the second aspect of the invention wherein the one or more biologically active cations is an anesthetic and the one or more biologically active anions is a coagulator, or wherein the one or more biologically active cations is a coagulator and the one or more biologically active anions is an anesthetic.

In another embodiment, the present invention provides compositions according to the second aspect of the invention wherein the one or more biologically active cations is an antibacterial and the one or more biologically active anions is a coagulator, or wherein the one or more biologically active cations is a coagulator and the one or more biologically active anions is an antibacterial.

In a specific embodiment, the present invention provides compositions according to the second aspect of the invention wherein the composition comprises benzalkonium piperacillin, didecyldimethylammonium piperacillin, or N-hexadecylpyridinium piperacillin.

In another specific embodiment, the present invention provides compositions according to the second aspect of the invention wherein the composition comprises lidocaine and docusate, miconazole/econazole and docusate, streptomycin and docusate, or isoniazide and docusate and lidocaine ibuprofenate, and lidocaine salicylate and lidocaine oleic acid and etodolac ibuprofenate.

In a further specific embodiment, the present invention provides compositions according to the second aspect of the invention wherein the composition comprises the cation benzalkonium and the anion comprises one or more of benzoate, colawet ma-80, fast green FCF, ibuprofen, penicillin G, piperacillin, docusate or sulfacetamide.

In a further specific embodiment, the present invention provides compositions according to the second aspect of the invention wherein the composition comprises as the cation, or first biologically active component, procaine or lidocaine and the anion, or second biologically active component, comprises one of more of aspirinate, cholate, decanoate, ibuprofenate, docusate, acetate, linoleate, niacinate, oleate, salicylate, acetylsalicylate, hexanoate, and stearate.

In another specific embodiment, the present invention provides compositions according to the second aspect of the invention wherein the composition comprises the cation choline and the anion comprises 5-fluorouracil, acyclovirate, ibuprofenate, or salicylate.

In another specific embodiment, the present invention provides compositions according to the second aspect of the invention wherein the composition comprises the cation, or first biologically active component, ephedrine and the anion, or second biologically active component, comprises cholate, decanoic acid, docusate, ibuprofenate, oleic acid, salicylate, or stearic acid.

In a further specific embodiment, the present invention provides compositions according to the second aspect of the invention wherein the composition comprises the cation hexadecylpyridinium and the anion comprises one or more of colawet ma-80, docusate, salicylate, fast green FCF, penicillin G, piperacillin, or sulfacetamide.

In another specific embodiment, the present invention provides compositions according to the second aspect of the invention wherein the composition comprises the anion docusate and the cations lidocainium, promethazinium, chlorpromazinium, ephedrinium, procainium, tramadolium, procainamidium, cetylpyridinium, benzalkonium, benzethonium, trihexyltetra-decylphosphonium, nicotinium, triclabendazolium, triclabendazolium sulfoxide, compound alpha, choline, mexilethinium, 5-aminolevulinic acid, ranitidine, silver ion, or mepenzolate.

In a third aspect, the present invention provides compositions according to the first aspect of the invention comprising at least one kind of cation and at least one kind of anion, wherein the composition is an ionic liquid that is liquid at a temperature at or below about 100° C., and wherein the at least one kind of cation, the at least one kind of anion, or both is a pesticidally active compound (i.e. a pesticide).

In another specific embodiment, the present invention provides compositions according to the first aspect of the invention wherein the biologically active cation is selected from a substituted sulfonium ion, a substituted phosphonium ion, a substituted ammonium ion, or mixtures thereof.

In specific individual embodiments, the present invention provides compositions according to the first aspect of the invention wherein the biologically active compound comprises one or more compounds selected solely from the compounds listed in Table 1, which have a melting or glass transition point at or below about 25° C.

In specific individual embodiments, the present invention provides compositions according to the first aspect of the invention wherein the biologically active compound comprises one or more compounds selected solely from the compounds listed in Table 2, which have a melting or glass transition point between about 25° C. and about 37° C.

In specific individual embodiments, the present invention provides compositions according to the first aspect of the invention wherein the biologically active compound comprises one or more compounds selected solely from the compounds listed in Table 3, which have a melting or glass transition point above about 37° C.

In specific individual embodiments, the present invention further provides compositions according to the first aspect of the invention wherein the biologically active compound comprises more than one compounds selected from the compounds listed in Table 1, Table 2 or Table 3, or from more than one of said tables.

In a fourth aspect, the present invention provides a pharmaceutical composition comprising a composition as defined by any of the other aspects of the invention.

The following tables further disclose biologically active compounds which may be supported on solid carrier material according to the present invention.

TABLE 1
biologically active compounds, oligomers, eutectic
and partially ionized compounds, having a melting
or glass transition point at or below about 25° C.

| Compound | Ratio |
|---|---|
| Procainamide oleic acid | 1:1 |
| Promethazine oleic acid | 1:1 |
| Promethazine stearic acid | 1:1 |
| Methyltributylammonium salicylate-salicylic acid | 1:2 |
| | 1:3 |
| Choline salicylate-salicylic acid | 1:2 |
| Cetylpyridinium salicylate-salicylic acid | 1:3 |
| Tetrabutylphosphonium ibuprofenate-ibuprofen | 1:2 |
| | 1:3 |
| Tetrabutylphosphonium lactate-lactic acid | 1:2 |
| | 1:3 |
| Lidocaine hexanoic acid | 1:1 |
| Lidocaine decanoic acid | 1:1 |
| Lidocaine oleic acid | 1:1 |
| Lidocaine linoleic acid | 1:1 |
| Procaine decanoic acid | 1:1 |
| Procaine oleic acid | 1:1 |
| Procaine linoleic acid | 1:1 |
| Tetrabutylphosphonium lactate | 1:1 |
| Ephedrine salicylate | 2:1 |
| excess base | 3:1 |
| Tramadolium decanoic acid | 1:1 |
| Tramadolium oleic acid | 1:1 |
| Ephedrinium clofibrate | |
| a) excess acid | 1:2 |
| b) excess base | 2:1 |
| Tetrabutylphosphonium salicylate-ibuprofen | 1:1:1 |
| Tetrabutylphosphonium salicylate-camphorsulfonic acid | 1:1:1 |
| Tetrabutylphosphonium salicylate-lactic acid | 1:1:1 |
| Tetrabutylphosphonium salicylate-cinnamic acid | 1:1:1 |
| Tetrabutylphosphonium ibuprofenate-niacin | 1:1:1 |
| Lidocaine ibuprofenate-salicylic acid | 1:1:1 |
| Cetylpyridinium salicylate - ibuprofenate | 1:1:1 |
| Cetylpyridinium salicylate - cinnamate | 1:1:1 |
| Cetylpyridinium salicylate - clofibrate | 1:1:1 |
| Ephedrinium-lidocaine salicylate | 1:1:1 |
| Tramadolium-lidocaine Ibuprofenate | 1:1:1 |
| Promethazine-ephedrinium docusate | 0.5:1:1 |
| | 1:1:1 |
| Promethazine-ephedrinium salicylate | 1:1:1 |
| Ephedrine decanoic acid | 1:1 |
| Ephedrine linoleic acid | 1:1 |
| Chlorpromazine oleic acid | 1:1 |
| Lidocaine docusate | 1:1 |
| Benzalkonium Ibuprofenate | 1:1 |
| Didecyldimethylammonium Ibuprofenate | 1:1 |
| Hexadecylpyridinium Ibuprofenate | 1:1 |
| Didecyldimethylammonium Saccharinate | 1:1 |
| Didecyldimethylammonium Acesulfamate | 1:1 |
| Ranitidine Docusate | 1:1 |
| Benzalkonium trans-cinnamate | 1:1 |
| Hexadecylpyridinium Colawet MA-80 | 1:1 |
| Benzalkonium Colawet MA-80 | 1:1 |
| Didecyldimethylammonium Colawet MA-80 | 1:1 |
| Didecyldimethylammonium Fast Green FCF | 1:1 |
| Lidocaine Ibuprofen | 1:1 |
| Lidocaine Sulfacetamide | 1:1 |
| Procaine Docusate | 1:1 |
| Procaine Ibuprofen | 1:1 |
| Procaine Salicylate | 1:1 |
| Tramadolium docusate | 1:1 |
| Lidocainium salicylate | |
| a) excess acid | 1:1.5 |
| | 1:2 |
| | 1:2.5 |
| | 1:3 |
| b) excess base | 2.5:1 |
| | 2:1 |
| | 1.5:1 |
| Benzethonium salicylate | 1:1 |
| Lidocainium acetylsalicylate | 1:1 |
| Tetrabutylphosphonium ibuprofenate | 1:1 |
| Tetrabutylphosphonium lactate | 1:1 |
| Promethazine docusate | 1:1 |
| Ephedrine docusate | 1:1 |
| 1-(2-(4-acetamidophenoxy)-2-oxoethyl)-3-methyl-1H- | 1:1 |
| imidazol-3-ium docusate | |
| 1-(2-(4-acetamidophenoxy)-2-oxoethyl)-1- | 1:1 |
| methylpyrrolidinium docusate | |
| (2-(4-acetamidophenoxy)-2-oxoethyl)tributyl-phosphonium | 1:1 |
| docusate | |
| 1-(2-(4-acetamidophenoxy)-2-oxoethyl)-3-methyl-1H- | 1:1 |
| imidazol-3-ium lactate | |
| 1-(2-(4-acetamidophenoxy)-2-oxoethyl)-1-methyl- | 1:1 |
| pyrrolidinium lactate | |
| Procainamide salicylate | 1:1 |
| Procainamide ibuprofenate | 1:1 |
| Procainamide docusate | 1:1 |
| Tributylhydroxyethylphosphonium docusate | 1:1 |
| Choline docusate | 1:1 |
| Tramadolium acetylsalicylate | 1:1 |
| Tramadolium cinnamate | 1:1 |
| Mexilethine docusate | 1:1 |
| Promethazine docusate | 1:1 |
| Chlorpromazine docusate | 1:1 |
| Trimethylhexadecyl-ammonium acyclovirate | 1:1 |
| Trimethylhexadecylammonium 5-fluorouracil | 1:1 |
| Triclabendazolium docusate | 1:1 |
| Cetylpyridinium docusate | 1:1 |
| Benzethonium docusate | 1:1 |
| Hexetidinium docusate | 1:1 |
| Trihexyltetradecylphosphonium docusate | 1:1 |
| Tetrabutylphosphonium artesunate | 1:1 |
| Lumefantrine artesunate | 1:1 |
| Nicotinium docusate | 1:1 |
| Benzalkonium Thimerosal | 1:1 |
| Hexadecylpyridinium Valproic Acid | 1:1 |
| Benzalkonium Mepenzolate Docusate | 1:1:2 |
| | 2:1:3 |
| | 1:2:3 |
| Benzalkonium Sulfathiazole Saccharinate | 2:1:1 |
| (2-acetoxyethyl)heptyloxymethyldimethyl-ammonium | 1:1 |
| Benzoate | |
| Didecyldimethylammonium PenicillinG | 1:1 |
| Didecyldimethylammonium Piperacillin | 1:1 |
| Didecyldimethylammonium Sulfacetamide | 1:1 |
| Benzalkonium docusate | 1:1 |
| 1-(2-(4-acetamidophenoxy)-2-oxoethyl)pyridinium docusate | 1:1 |
| Choline acyclovirate | 1:1 |
| Tetrabutylphosphonium salicylate | 1:1.2 |
| | 1:1.3 |
| | 1:1.4 |
| | 1:1.5 |
| | 1:1.6 |
| | 1:1.7 |
| | 1:1.8 |
| | 1:1.9 |
| | 1:2 |
| | 1:2.4 |
| | 1:2.7 |
| | 1:2.8 |
| | 1:3 |
| | 1:3.1 |
| | 1:3.5 |
| | 1:3.7 |
| Dicamba Choline | 1:1 |
| Trihexylalkylphosphonium Dicamba | 1:1 |
| Benzalkonium Dicamba | 1:1 |
| Compound alpha docusate | 1:1 |
| Triclabendazolium sulfoxide docusate | 1:1 |
| Benzethonium artesunate | 1:1 |
| Tributylmethylammonium acyclovirate | 1:1 |
| Benzethonium acyclovirate | 1:1 |

TABLE 2
biologically active compounds, oligomers, eutectic and
partially ionized compounds, having a melting or glass
transition point between about 25° C. and about 37° C.

| Compound | Ratio |
|---|---|
| Lidocainium acetic acid | 1:1 |
| Ephedrine oleic acid | 1:1 |
| Didecyldimethylammonium Salicylate | 1:1 |
| Hexadecylpyridinium Acesulfamate Saccharinate | 2:1:1 |
| | 3:2:1 |
| Benzalkonium Penicillin G | 1:1 |
| Hexadecylpyridinium Piperacillinate | 1:1 |
| Benzalkonium Piperacillinate | 1:1 |
| Hexadecylpyridinium Sulfacetamide | 1:1 |
| Hexadecylpyridinium Acesulfamate Saccharinate | 3:1:2 |
| Benzalkonium Acesulfamate Saccharinate | 3:1:2 |
| | 3:2:1 |
| Benzalkonium Sulfathiazole Saccharinate | 3:1:2 |
| | 3:2:1 |
| Hexadecylpyridinium Penicillin G | 1:1 |
| Benzalkonium Sulfacetamide | 1:1 |
| Benzalkonium Fast Green FCF | 1:1 |

TABLE 3
biologically active compounds, oligomers, eutectic
and partially ionized compounds, having a melting
or glass transition point above about 37° C.

| Compound | Ratio |
|---|---|
| Cetylpyridinium salicylate-salicylic acid | 1:2 |
| Lidocaine stearic acid | 1:1 |
| Lidocainium salicylate | |
| a) excess base | 3:1 |
| Procaine stearic acid | 1:1 |
| Hexadecylpyridinium Sulfathiazole | 1:1 |
| Tramadolium stearate | 1:1 |
| Ephedrinium-lidocaine Ibuprofenate | 1:1:1 |
| Tramadolium-lidocaine Salicylate | 1:1:1 |
| Ephedrine stearic acid | 1:1 |
| Ephedrine salicylate | |
| a) excess acid | 1:1.1 |
| | 1:1.2 |
| | 1:1.3 |
| | 1:1.4 |
| | 1:2 |
| | 1:3 |
| | 1:4 |
| Benzalkonium Saccharinate | 1:1 |
| Hexadecylpyridinium Saccharinate | 1:1 |
| Benzalkonium Acesulfamate | 1:1 |
| Hexadecylpyridinium Acesulfamate | 1:1 |
| Hexadecylpyridinium Fast Green FCF | 1:1 |
| 3-hydroxy-1-octyloxymethylpyridinium Saccharinate | 1:1 |
| Didecyldimethylammonium trans-Cinnamate | 1:1 |
| Benzalkonium Sulfathiazole | 1:1 |
| Mepenzolate docusate | 1:1 |
| Benzalkonium Acesulfamate Saccharinate | 2:1:1 |
| 3-hydroxy-1-octyloxymethylpyridinium Acesulfamate | 1:1 |
| Tramadolium rac-ibuprofenate | 1:1 |
| Tramadolium meclofenamate | 1:1 |
| Cetylpyridinium salicylate | 1:1 |
| Ephedrine salicylate | 1:1 |
| Tetrabutylphosphonium salicylate | 1:1 |
| | 1:1.1 |
| Methyltributylammonium salicylate | 1:1 |
| Triethanolammonium ibuprofenate | 1:1 |
| Benzalkonium salicylate | 1:1 |
| Hexetidinium ibuprofenate | 1:1 |
| Quinine artesunate | 1:1 |
| 5-aminolevulinic docusate | 1:1 |
| Choline 5-fluorouracil | 1:1 |
| Tetrabutylphosphonium 5-fluorouracil | 1:1 |

The invention will be exemplified by the following non-limiting examples.

### EXAMPLES

### Example 1

### Synthesis of Choline Acyclovir [1]

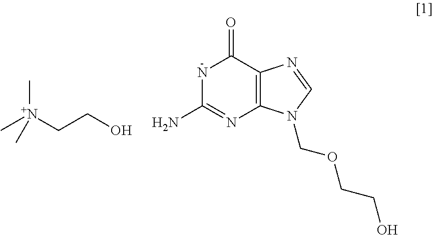

Acyclovir (0.693 mg, 3 mmol) was suspended in 20 ml of ethanol and a 46% solution of choline hydroxide in water (3 mmol) was added dropwise. The suspension was stirred for 15 min at room temperature until a clear solution was obtained and evaporated. Remaining volatile material was removed under reduced pressure (0.01 mbar, 50° C.) to yield choline acyclovir [3] as colourless glass.

¹H-NMR (300 MHz, d₆-DMSO) δ (ppm)=7.4 (s, 1H), 5.2 (s, 2H), 4.9 (br s, 2H), 3.8 (s, 2H), 3.4 (m, 6H), 3.0 (s, 9H). ¹³C-NMR (75 MHz, d₆-DMSO) δ (ppm)=167.9, 161.8, 134.5, 118.9, 71.9, 70.4, 67.7, 60.3, 55.6, 53.5.

### Example 2

### Synthesis of Tributylmethylammonium Acyclovir [2]

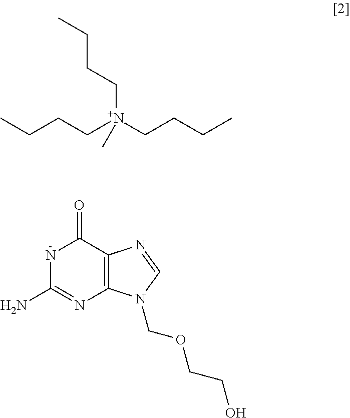 [SMILES: CCCC[N+](C)(CCCC)CCCC ; Nc1nc2c(ncn2COCCO)c(=O)[nH]1]

Prepared according to example 1 to give tributylmethylammonium acyclovir [2] as colourless solid.

¹H-NMR (300 MHz, d₆-DMSO) δ (ppm)=7.5 (s, 1H), 5.3 (s, 2H), 3.5 (s, 4H), 3.2 (m, 7H), 2.9 (s, 3H), 1.6 (m, 6H), 1.4 (m, 6H), 0.9 (m, 9H).

### Example 3

### Synthesis of Trimethylhexadecylammonium Aciclovir [3]

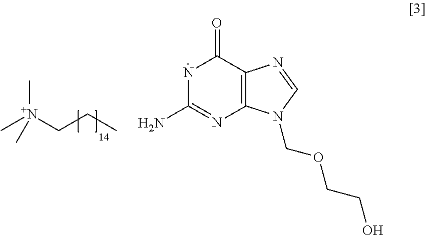

Prepared according to example 1 to give trimethylhexadecylammonium acyclovir [3] as colourless solid.

### Example 4

### Synthesis of Dioctylsulfosuccinic Acid [4]

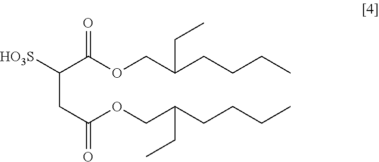 [SMILES: CCCCC(CC)COC(=O)CC(C(=O)OCC(CC)CCCC)S(=O)(=O)O]

Silver docusate¹ (10 g, 18.89 mmol) was suspended in 30 ml of methanol and HCl (37% solution in water; 1.56 mL, 18.89 mmol) was added dropwise. The suspension was stirred overnight at room temperature. The precipitate was filtered through Celite® and the filter cake was washed with additional 10 mL of cold methanol. The solvent was removed under reduced pressure (0.01 mbar, 50° C.) to yield dioctylsulfosuccinic acid quantitatively, as a light yellow viscous liquid. ¹ Rogers et al. “Multi-functional ionic liquid compositions for overcoming polymorphism and imparting improved properties for active pharmaceutical, biological, nutritional, and energetic ingredients”, US 20070093462, Apr. 26, 2007

¹H-NMR (300 MHz, d₆-DMSO) δ (ppm)=6.16 (br), 3.93-3.62 (m, 4H), 3.56 (s, 1H), 3.28 (d, 1H), 2.94-2.77 (m, 2H), 1.50 (br, 2H), 1.24 (br, 16H), 0.83-0.81 (m, 12H).

### Example 5

### Synthesis of Itraconazolium Dioctylsulfosuccinate [5]

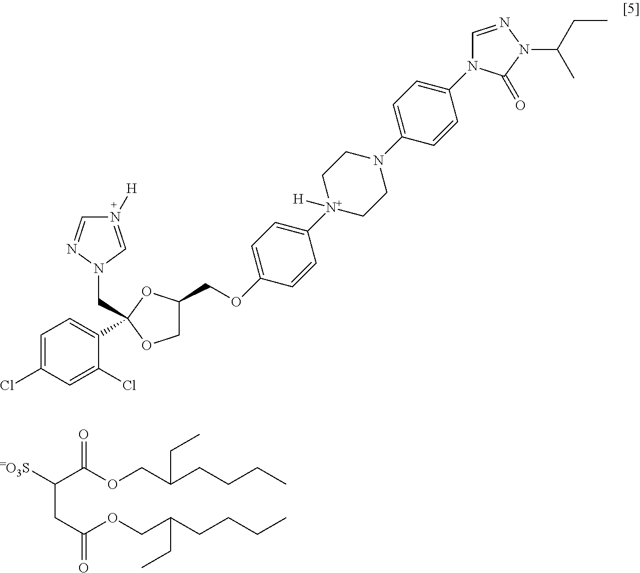

Dioctylsulfosuccinic acid (3.752 g, 8.88 mmol) was suspended in 10 mL acetone and itraconazole (3.135 g, 4.44 mmol) was added in small portions. With itraconazole addition, the solution changed its color from light yellow, to green, and after overnight stirring to light orange. The volatiles were removed under reduced pressure, and the resulted viscous material was further dried (0.01 mbar, 60° C.), to yield 6.8 g of itraconazolium dioctylsulfosuccinate as a light brown glass.

### Example 6

### Synthesis of Tetraethylammonium Glyphosate [6]

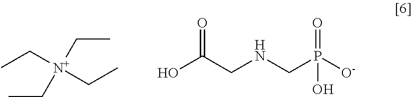

Tetraethylammonium chloride (1.66 g, 100 mmol) was suspended in 20 mL distilled water and NaOH (0.44 g, 110 mmol) dissolved in distilled water was added dropwise. AgNO3 solution (1.7 g, 100 mmol dissolved in 20 mL distilled water) was added and the resulting mixture was stirred at 50° C. for 20 minutes. After cooling, the obtained solid was filtered and washed with distilled water. At this point, glyphosate (1.7 g, 100 mmol) was added and the reaction mixture was stirred at room temperature for 14 hours. Water was removed using a rotary evaporator and the obtained product was dried under reduced pressure at 60° C. for 24 hours.

¹H-NMR (300 MHz, D₂O) δ (ppm)=4.9 (s, 3H), 3.73 (s, 2H), 3.28 (d, J=12.8 Hz, 2H), 3.22 (q, J=7 Hz, 8H), 1.25 (t, J=9.1 Hz, 12H). ¹³C-NMR (125 MHz, D₂O) δ (ppm)=173.6, 54.7, 47.8, 46.0, 9.4.

### Example 7

### Synthesis of Tetrabutylphosphonium Ibuprofenate [7]

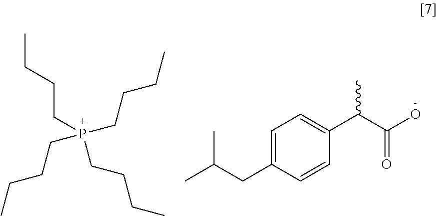

Ibuprofenic acid (1.032 g, 5 mmol) and tetrabutylphosphonium hydroxide (˜40% sol. in H₂O) (3.414 g, 5 mmol) were dissolved in 20 mL of acetone stirred for 15 min at room temperature. The solvent was evaporated and the remaining viscous liquid was dried at 0.1 mbar with stirring for 24 hours to obtain tetrabutylphosphonium ibuprofenate [7] in quantitative yield as a colourless viscous liquid. ¹H-NMR (300 MHz, d₆-DMSO) δ (ppm)=7.13 (d, J=8.08 Hz, 2H), 6.94 (d, 8.08 Hz, 2H), 3.21 (q, 7.74 Hz, 1H), 2.48 (m, 2H), 2.36 (d, 7.28 Hz, 2H), 2.14 (m, 8H), 1.77 (sept, 6.15 Hz, 1H), 1.40 (m, 16H), 1.18 (d, J=7.03 Hz, 3H), 0.91 (t, 7.02 Hz, 12H), 0.84 (d, J=7.02 Hz, 6H). ¹³C-NMR (75 MHz, d₆-DMSO) δ (ppm)=174.8, 144.2, 136.9, 127.8, 127.2, 49.3, 44.4, 29.7, 23.4 (d, J=15.8 Hz), 22.7 (d, J=4.7 Hz), 22.2, 20.5, 17.3 (d, J=48.1 Hz), 13.3. IR (neat) v=2957, 2929, 2870, 1588, 1459, 1371, 1341, 860, 721 cm⁻¹. HRMS (ES+) [m/z]=259.2550; (ES−) [m/z]=205.1237.

T_{g} −43° C., T_{5%onset} 237° C.

### Example 8

### Synthesis of Silica-Supported Tetrabutylphosphonium Ibuprofenate [7a]

Tetrabutylphosphonium ibuprofenate [7] (0.400 g, 0.86 mmol) and mesoporous silica-90 (Silica gel 90 (Fluka); particle size 0.063-0.200 mm, BET surface area 298 m²/g; total porosity 1.02 cm³/g, 1.6 g) were suspended in 20 mL of anhydrous ethanol and stirred at room temperature for 2 hours. The solvent was slowly evaporated and remaining volatile material was removed under reduced pressure (0.01 mbar, 50° C.) to yield silica-supported tetrabutylphosphonium ibuprofenate [7a] as off-white solid.

IR (neat) v=3668, 2953, 2870, 1571, 161, 1384, 1055, 800 cm⁻¹.

### Example 9

### Synthesis of Lidocainium Ibuprofenate [8]

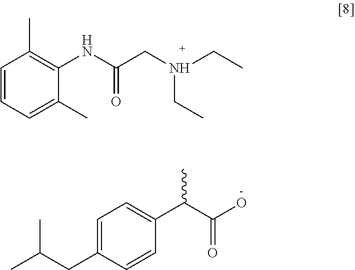

Ibuprofenic acid (2.343 g, 15 mmol) and lidocaine (3.094 g, 15 mmol) were melted in a sealed vial with stirring until a free-flowing clear liquid was obtained. The mixture was cooled to room temperature to obtain [8] as a colourless clear liquid in >99% yield.

¹H-NMR (300 MHz, d₆-DMSO) δ (ppm)=9.19 (s, 1H), 7.19 (d, J=8.03 Hz, 2H), 7.08 (m, 5H), 3.63 (q, J=7.07 Hz, 1H), 2.63 (q, J=6.87 Hz, 4H), 2.42 (d, J=7.47 Hz, 2H), 2.15 (s, 6H), 1.81 (m, 1H), 1.35 (d, J=7.38 Hz, 2H), 1.08 (t, J=7.16 Hz, 6H), 0.86 (d, 6.74 Hz, 2H). ¹³C-NMR (75 MHz, d₆-DMSO) δ (ppm)=175.5, 169.4, 139.5, 138.6, 153.2, 135.1, 129.0, 127.6, 127.1, 126.2, 56.8, 48.1, 44.4, 44.3, 29.7, 22.2, 18.6, 18.2, 12.1. IR (neat) v=3268, 2964, 2931, 2871, 1680, 1501, 1461, 1379, 1209, 1065, 768 cm⁻¹. HRMS (ES+) [m/z]=235.1799; (ES−) [m/z]=205.1249.

Tg −27° C., T_{5%onset} 177° C.

### Example 10

### Synthesis of Silica-Supported Lidocainium Ibuprofenate [8a]

Prepared from lidocainium ibuprofenate [8] (0.400 g, 0.91 mmol) and SiO₂-90 (Silica gel 90 (Fluka), 1.6 g) according to example 8 to yield silica-supported lidocainium ibuprofenate [8a] as white solid.

### Example 11

### General Synthesis of Silica-Supported Compounds

Silica was dried under heating (70° C.) and vacuum (0.01 mbar). API-IL (or starting API) was dried under vacuum and heated to remove volatiles or water and then weighed out ca. 0.01 g, and dissolved in suitable dry solvent (dry acetone or purchased anhydrous methanol or ethanol) to complete dissolution (˜20 mL of solvent). Silica-SiO₂-90 (appropriate to target loading) was suspended in solvent with dissolved API in it (20 mL) and stirred for 2 h at rt. The solvent was evaporated (Rotovap) and sample kept under high vacuum (0.01 mbar) overnight.

### Example 12

### Controlled Release of Silica-Supported Tetrabutylphosphonium Ibuprofenate [7] (FIG. 10)

100 mg of SILP was suspended in 100 mL of preheated media (phosphate buffered saline, simulated gastric fluid or simulated intestinal fluid according to USP standards) and placed in a thermostated shaker at 37° C. with 150 rpm. In intervals, a 250 μL sample was taken and diluted to 2.5 mL, filtered over a syringe filter to stop the leaching, and measured via UV-visible spectrometry. 250 μL of fresh media were immediately added to the leaching experiment to replace the missing volume.

TABLE 4
Thermal stability determination
Thermal stability for the compounds was measured by determining the
inflection point using a TA2950 TGA unit by heating from 25° C. to
800° C. with a heating rate of 5° C./min under air except for
Ibuprofene and [7] which were recorded under nitrogen and
where the T_{5% onset} temperature was measured instead.

| | | | Inflection |
|---|---|---|---|
| | Fig- | | point |
| Compound | ure | Loading | [° C.] |
| Acyclovir | 1 | not supported/ | 249 |
| | | neat | |
| Acyclovir on SiO₂ | | 10% | 257 |
| Acyclovir on SiO₂ | | 20% | 260 |
| Acyclovir on SiO₂ | | 50% | 270 |
| Choline Acyclovir [1] | 2 | not supported/ | 123 |
| | | neat | |
| [1] on SiO₂ | | 10% | 167 |
| [1]on SiO₂ | | 20% | 165 |
| Tributylmethylammonium Acyclovir | 3 | not supported/ | 203 |
| [2] | | neat | |
| [2] on SiO₂ | | 10% | 208 |
| [2] on SiO₂ | | 20% | 204 |
| Trimethylhexadecylammonium | 4 | not supported/ | 189 |
| acyclovir [3] | | neat | |
| [3] on SiO₂ | | 10% | 241 |
| [3] on SiO₂ | | 20% | 234 |
| Dioctylsulfosuccinic Acid [4] | 5 | not supported/ | 162 |
| | | neat | |
| [4] on SiO₂ | | 10% | 233 |
| [4] on SiO₂ | | 20% | 218 |
| Itraconazolium Dioctylsulfosuccinate | 6 | not supported/ | 257 |
| [5] | | neat | |
| [5] on SiO₂ | | 10% | 280 |
| [5] on SiO₂ | | 20% | 266 |
| Tetraethylammonium Glyphosate [6] | 7 | not supported/ | 150 |
| | | neat | |
| [6] on SiO₂ | | 10% | 179 |
| [6] on SiO₂ | | 20% | 176 |
| Ibuprofene | 8 | not supported/ | 155* |
| | | neat | |
| Ibuprofene on SiO₂ | | 10% | 300* |
| Tetrabutylphosphonium | 9 | not supported/ | 236* |
| Ibuprofenate [7] | | neat | |
| [7] on SiO₂ | | 10% | 386* |
| [7] on SiO₂ | | 20% | 263* |

*T_{5% onset} temperature was measured instead of inflection point

1. A composition comprising a biologically active compound which is non-covalently and releaseably adsorbed on a solid carrier material consisting of mesoporous silica, wherein by placement in an aqueous environment said biologically active compound is released from said carrier material.

2. The composition according to claim 1, wherein the biologically active compound comprises one or more compounds and mixtures thereof.

3. The composition according to claim 2, wherein the biologically active compound comprises mixtures of ionic and non-ionic compounds.

4. The composition according to claim 1, wherein the biologically active compound comprises one or more biologically active ions.

5. The composition according to claim 1, wherein the biologically active compound is a eutectic mixture comprising one or more biologically active compounds.

6. The composition according to claim 1, wherein the biologically active compound is non-ionic.

7. The composition of claim 1, wherein the biologically active compound is liquid at or below 25° C.

8. The composition of claim 1, wherein the biologically active compound is liquid at or below 37° C.

9. The composition of claim 1, wherein the biologically active compound is liquid above 37° C.

10. The composition according to claim 12, wherein by placement in an aqueous environment both the anionic and cationic parts of said ionic compound are released from said carrier material.

11. The composition of claim 4, wherein the one or more biologically active ions are one or more biologically active cations and/or one or more biologically active anions.

12. The composition according to claim 11, wherein the one or more biologically active ions are one or more biologically active cations and one or more biologically active anions.
